(19) United States
(12) Patent Application Publication
(10) Pub. No.: US 2024/0399128 A1
(43) Pub. Date: Dec. 5, 2024

(54) COSMETIC DEVICE

(71) Applicant: Sharon HILDITCH, Liverpool Merseyside (GB)

(72) Inventor: Sharon HILDITCH, Liverpool Merseyside (GB)

(21) Appl. No.: 18/698,999

(22) PCT Filed: Oct. 6, 2022
(86) PCT No.: PCT/GB2022/052523

§ 371 Date: Apr. 5, 2024

(30) Foreign Application Priority Data
Oct. 6, 2021 (GB) 2114313.6

(51) Int. Cl. A61M 35/00; A61F 7/00; A61M 11/00

(52) U.S. Cl. CPC A61M 35/30; A61F 7/007; A61M 11/00; A61F 2007/0063; A61F 2007/0076; A61M 2202/0208

(57) ABSTRACT

The present invention provides a liquid cooling and oxygenation device comprising: a container for holding a liquid; a cooling means for cooling the liquid in the container to a temperature of less than about 15° C.; a means for supplying an oxygen-enriched gas to the cooled liquid in the container; and a means for dispensing the cooled, oxygenated liquid.

### FIELD OF THE INVENTION

The present application relates to a liquid cooling and oxygenation device, and more particularly to a cosmetic skin treatment device, and, in particular, the device being able to for dispense a cooled, oxygenated liquid, which can be used for cosmetically treating the skin of a person to improve the appearance of the skin or for other applications.

### BACKGROUND

The use of oxygen in therapeutic skin treatment has been known for some time. Oxygen is applied onto the skin at high pressure in order to improve circulation and cell regeneration. This technique can be used in combination with beauty care products, usually in the form of a cream, gel or lotion, that is rubbed into the outer layer of the skin (the epidermis). The high-pressure oxygen is then used to then force the cream or lotion through the epidermis.

However, there is a need for a simpler way of applying oxygen to the skin while still achieving good oxygen penetration through the skin, as the provision of high-pressure oxygen to the skin requires high amounts of energy, as well as specialist equipment.

### STATEMENTS OF INVENTION

In accordance with a first aspect of the present invention, there is provided a liquid cooling and oxygenating device comprising:
a container for holding a liquid;
cooling apparatus for cooling the liquid in the container to a temperature of less than about 15° C.;
an arrangement to supply an oxygen-enriched gas to the cooled liquid in the container; and
an outlet arrangement for dispensing the cooled, oxygenated liquid.

By cooling the liquid that is to be used, which may be for consumption or for application to skin, such as a serum or liquid, the concentration of oxygen contained therein can be increase, compared with the liquid being at higher temperatures.

In respect of application to skin during, for example, cosmetic treatment, device may be a cosmetic skin treatment device that can cool and oxygenate a liquid, wherein the increased concentration of oxygen that is dissolved within the liquid allows for a deeper penetration of oxygen into the skin than high-pressure gaseous oxygen. Thus, oxygen can penetrate both the epidermis and the dermis without requiring the application of high-pressure oxygen to the skin, thereby providing a more intense treatment to rejuvenate skin texture and firmness. This provision of topical oxygen solution being applied to the skin of a person provides a deep penetration of oxygen to the layers of the skin, that may improve the appearance and health of the skin.

Thus, a cosmetic skin treatment device according to the present invention can provide a cosmetic liquid, such as a gel or a serum, with an increased amount of dissolved oxygen therein and an improved oxygen retention rate. Thus, deep oxygen penetration of the skin is possible without requiring the separate application of high-pressure oxygen to the skin. It will be appreciated that the cooled, oxygenated liquid, or liquids, of the present invention can also be dispensed in combination with high-pressure oxygen in order to further improve the penetration of oxygen through the layers of the skin.

The device according to the present invention provides an increased flexibility in the storage and application of one or more liquids, particularly cosmetic liquids, without the need for different, separate devices and cooling means. In cosmetic applications, if the operator wishes to apply different liquids at different temperatures and different levels of oxygenation, this could be achieved by way of a device according to the present invention, wherein the device comprises a plurality of containers, each attached to cooling apparatus, which may be individual or a single cooling apparatus cooling a plurality of liquids, thereby reducing and/or regulating the temperature of the liquid(s) and, so the degree of oxygen contained therein.

In respect of beverages or other applications, the increased level of oxygen in the liquid may have further benefits and/or may be useful in providing oxygenated liquid for a particular purpose. For example, it has been suggested that ingesting oxygen may help with exercise recovery by increasing lactate clearance.

The term “oxygen-enriched gas” is intended to refer to a gas that has a higher oxygen level than ambient air.

It is advantageous that the device of the present invention is portable, which may be through the use of wheels or castors. This allows the device to be readily transported to a location where it can be used and, in cosmetic or beverage applications, it can be easily moved to the next customer. This is particularly useful because transporting the oxygenated liquid after it is dispensed may result in the dissolved oxygen dissipating into the surrounding ambient air. Thus, by making the device portable, a better concentration of oxygen will be present in the dispensed cooled and oxygenated liquid.

Preferably, the cooling apparatus for cooling the liquid in the container may be arranged to cool the liquid to a temperature of less than about 10° C. and, preferably, the cooling means may be arranged to cool the liquid to a temperature between about 0 and 7° C. When using a liquid having a temperature of less than about 10° C., and preferably between 0 and 7° C., the solubility of oxygen in the liquid is increased, thereby increasing the amount of oxygen that can be dissolved in the liquid. Therefore, the resulting cooled, oxygenated liquid has a high dissolved oxygen content, or concentration, which is beneficial for the treatment of a patient's skin. The aforementioned temperatures have also been found to improve the oxygen transfer retention rate of the liquid, compared to conventional processes in the art. The oxygen transfer retention rate is the rate at which dissolved oxygen is retained within the liquid.

Thus, the oxygenation of a cooled liquid and the use thereof according to the present invention increases the amount of dissolved oxygen in the liquid by increasing both solubility and oxygen retention. This may allow the liquid and the dissolved oxygen to penetrate through deeper layers of the patient's skin than conventional techniques. This may help to rejuvenate and rehydrate the skin, reducing the appearance of lines and wrinkles, giving the skin a more youthful appearance. Additionally, the use of the oxygenated liquid can reduce the appearance of certain skin conditions.

In one arrangement, the container of the present invention may have a cap, or other lid element, that can be removed so that a liquid can be placed in the container. The cap may be vented to allow the oxygen-enriched air to escape from the container. The cap may reduce the risk of contaminants entering the liquid and assists with keeping the liquid at in the cooled temperature state. The cap may be releasably attached by conventional means, including screwing, push-fitting or snap-fitting the cap to the container.

Advantageously, the means for supplying the oxygen-enriched gas to the cooled liquid in the container may comprise a gas tip, which can extend into the container. Additionally, or alternatively, the means for supplying the oxygen-enriched gas may comprise a gas diffuser, which may be a sintered diffuser. The gas tip and/or gas diffuser may be made from stainless steel.

The gas tip may extend from a top portion of the container, that is, from the end of the container adjacent the cap and/or, the gas tip may extend from the cap. The gas diffuser is, preferably, positioned in a bottom portion of the container, that is to say, the opposite end of the container to the cap and/or the lowermost part of the container. In an advantageous arrangement, the gas diffuser covers all or part of the base of the container. This allows a large area through which the oxygen can enter the liquid within the container.

In a preferred embodiment, the oxygen-enriched gas is supplied in the form of fine gas bubbles that enter into the liquid. Thus, the gas diffuser and/or gas tip promotes aeration via the efficient transfer of fine bubbles into the liquid, which increases the dissolved oxygen content in the liquid.

The arrangement of the present invention allows the liquid in the container to be simultaneously cooled and oxygenated, thereby increasing the amount of dissolved oxygen in the liquid due to both increased solubility and retention.

Oxygen-enriched gas may be supplied from an oxygen concentrator. The oxygen concentrator concentrates the oxygen from a gas supply, for example ambient air, by selectively removing nitrogen to provide an oxygen-enriched product gas stream. The oxygen-enriched product gas stream can be at atmospheric pressure or a low-pressure oxygen-enriched gas stream. Thus, high-pressure oxygen flow is not required.

The oxygen-enriched gas is, preferably, at least 80%, and more preferably, at least 85%, or at least 90% oxygen.

A peristaltic pump, or other arrangement, may be used to transfer the liquid from the container through a tube into a handset, whereafter it can be dispensed onto the skin of a patient. The handset may be supplied with a compressed air outlet to infuse the liquid into the layers of the skin.

It is preferable that the cooling apparatus that is employed for cooling the liquid comprises a cooling block that is in thermal contact with a water bath in which the container is positioned. The cooling block may be in direct thermal contact with the water in the water bath, thereby cooling the water and, in turn, cooling the liquid within the container. The water bath may be attached to the cooling block by, for example, screwing or snap-fitting the water bath into the cooling block. The water bath may, itself be provided with a lid or cap arrangement, or the lid may be common to both the container and the water bath. It will be appreciated that the container may be held within the water bath without a cap, although this is inefficient. Similarly, the container may be held in the water bath using a support element, with the contain passing through the lid or cap of the water bath.

The term “thermal contact” as used herein is intended to mean that heat can flow between the two components, either directly or via one or more intermediate components. The term “direct thermal contact” as used herein means that heat can flow directly between the two components, without any intermediate components.

The water within the water bath can be cooled by transferring heat efficiently from water in the water bath to the cooling block. Thus, in this embodiment, the liquid in the container is not in direct thermal contact with the cooling means; however, this arrangement allows the liquid in the container to be cooled from all of the surfaces that are in contact with the water in the water bath, thereby resulting in a more uniform low temperatures being achieved in the container.

In an embodiment where the cooling means for cooling the liquid in the container comprises a water bath and a cooling block, the cooling block is preferably made from metal, such as aluminium, or another conductive material. It will be appreciated that an aluminium cooling block in an arrangement according to the present invention provides a good degree of heat transfer, thereby efficiently allowing the device of the present invention reaching and maintaining the desired temperature.

It is envisaged that in some arrangements, the cooling block will freeze a portion of the water in the water bath adjacent to the cooling block. The frozen portion of the water may be a solid ring of ice. Which can be 1 cm thick. The ice is, preferably, not in contact with the container in the water bath in order to avoid freezing the liquid therein and to maintain a more constant temperature in the container.

The water bath may comprise a water bath cap on the end of the water bath opposite the cooling block. The container for holding the liquid may be attached to said cap, such that the cap of the water bath and the cap of the container are the same component. The cap may be vented.

The container may be screwed onto the water bath cap. The bottom of the container, with the liquid therein, is positioned above the cooling plate, for example, approximately 2 cm above the cooling plate, although it will be appreciated that the cooling plate may be arcuate or arranged adjacent the side of the water bath in some embodiments.

The supply of oxygen-enriched gas to the cooled liquid may be delivered by way of a gas tip that extends into the container from the cap of the water bath. Thus, the gas tip can extend from a top portion of the container into the liquid in the container.

In a particular embodiment, the cooling apparatus for cooling the liquid comprises a cooling block that is positioned to be in direct thermal contact with the liquid. The cooling block therefore may be in direct contact with the liquid in the container. In this arrangement, the cooling block is preferably made from metal, such as stainless steel. The container may be attached to the cooling block by any conventional means, such as screwing the container into the cooling block. It may be preferred that the cooling block is in contact with the container, rather than the liquid directly. In this arrangement, a larger amount of liquid can be cooled but as only part of the liquid is in contact with the cooling block, higher temperatures are generally reached compared to the embodiment including a water bath. Additionally, an agitation mechanism to mix the liquid may be desirable to ensure a more even cooling of the liquid.

The supply of oxygen-enriched gas to the cooled liquid, and the elements therefor, may be include, or may be a gas diffuser located within the container. The gas diffuser may be positioned at the bottom portion, or lowermost portion, of the container and, preferably, the diffuser covers part, but not all, of the cooling block.

Advantageously, the cooling apparatus comprises a thermoelectric cooler module, and it is particularly advantageous that the cooling apparatus comprises a Peltier module or an element that operates to produce a Peltier effect. The cooling apparatus may also comprise a heat sink. The thermoelectric cooler module may drive the transfer of heat from the liquid in the container, through the cooling block and, optionally. to a heat sink.

In one arrangement, the heat sink may be in direct thermal contact with the thermoelectric cooler module. Additionally, or in an alternative arrangement, there may be thermally conductive gap fillers on one or both sides of the thermoelectric cooler module. There may be thermally conductive gap fillers on both sides of the thermoelectric cooler module. This can improve the efficiency of heat transfer from the cooling block, through the thermoelectric cooler module and to the heat sink, thereby improving the efficiency of cooling the liquid in the container.

The cooling block may be surrounded by an insulating material, such as an insulation foam. The use of an insulator to thermally insulate the cooling block from the ambient air, increases the efficiently of the apparatus in cooling the liquid.

The device of the present invention may further comprise an air circulation device configured to flow air past the heat sink. This helps to dissipate heat, thereby maintaining the cooling efficiency of the cooling means.

The cooling block may be made from metal or a metal alloy, such as stainless steel or aluminium. The material can be selected based on the desired temperature of the liquid in the container, as aluminium is more thermally conductive than stainless steel, it will cool the liquid to a lower temperature.

The liquid may be drawn through the cooling block prior to application to the skin, as it is removed from the container. This is particularly applicable when the cooling block is in direct thermal contact with the liquid because this further reduces the temperature of the liquid, thereby improving the oxygen retention rate during the dispensing process.

As mentioned above, the device according to the present invention can have a plurality of containers for holding liquid, each of which can be associated with a separate cooling means for cooling the liquid to a temperature of less than about 15° C. and each potentially having a separate oxygen supply system for supplying an oxygen-enriched gas to the cooled liquid. The plurality of containers and each of their cooling means and means for supply an oxygen-enriched gas may each have any of the features discussed above. Similarly, the features discussed above my apply where a single cooling system is employed for a plurality of containers.

The cooling apparatus and/or the oxygen-enriched gas supply arrangement to each of the containers may be the same for a plurality of containers or may be individual. In one embodiment, the cooling apparatus and/or the oxygen-enriched gas supply system of a first container is different to that of a second container.

Thus, the present invention may be in the form of a modular arrangement wherein further containers, with associated cooling apparatus and oxygen-enriched gas supply arrangements, allows for flexibility in the arrangement and for an ability to control the temperature, because adjacent containers can contain liquids that are applied in sequence or in parallel but that are provided at different temperatures and with different oxygen concentrations.

In one embodiment, the cooling apparatus of the first container comprises a cooling block that is positioned to be in direct thermal contact with the liquid and the cooling means of the second container comprises a cooling block that is in thermal contact with a water bath in which the container is positioned. Each of these containers can have any of the features discussed above. As a result, of the arrangement of the cooling block being in thermal contact with the liquid in the containers, the temperature of the liquid in the first container can be different to the temperature of the liquid in the second container. In other words, the cooling apparatus of the first container can cool a liquid to a different temperature than the cooling apparatus of the second container. Furthermore, it will be appreciated that where the cooling apparatus is common to more than one container, the temperatures of the respective containers may be different in each by varying the cooling effect acting on the containers.

In the arrangement discussed above, the cooling apparatus of the first container may cool the liquid to between 0 and 8° C., between 2 and 7° C., or between 3 and 6° C. and/or the cooling means of the second container may cool the liquid to between 0 and 5° C., between 0 and 3° C., or between 1 and 2° C. This increases the flexibility of the device, as the different cooling apparatus can be selected to create oxygenated liquids having different temperatures and oxygen levels.

Each of the cooling means be provided with a separate heat sink, as discussed above, although it will be appreciated that a common heat sink may be advantageous in some circumstances. An air circulation device may be configured to flow air past the, or each, of the heat sinks, thereby dissipating heat from the, or each of, the heat sink in an efficient manner. The container in which a higher temperature liquid is held may be further along the air flow than a container in which a lower temperature liquid is held.

The cooling block of each container may be attached to a support, such as a manifold. This can be by any conventional means, such as screwing or snap-fitting the cooling block into place. The container(s) and/or water bath(s) may then be attached to the cooling blocks by any conventional means, such as screwing or snap-fitting.

The oxygen-enriched gas may be cooled before it is supplied to the one or more containers of the device. The gas is cooled to below 20° C., preferably below 10° C., thereby improving the cooling efficiency and oxygen dissolution in the liquid. This can be achieved by employing an oxygen concentrator, which can concentrate oxygen present in the ambient air. Compressor. Alternatively, or additionally, a replaceable gas cylinder containing oxygen may be fitted to the device. The oxygen-enriched gas may then be cooled by a cooling system, which may include a Peltier element.

In one arrangement, the oxygen-enriched gas may comprise at least about 80%, at least about 85%, or at least about 90% oxygen. Supplying highly-concentrated oxygen to the liquid increases the amount of oxygen that is dissolved therein.

The liquid in the container may be any cosmetic liquid, such as a gel or a serum and the liquid may be a cleanser, a peel or a microdermabrasion product. A powdered cosmetic may be mixed with water or another liquid to form a liquid for use with the device described above. Similarly, the liquid in the container may be a consumable, such as water or alcoholic spirits, or it may be employed for the treatment of injuries, for example, by physiotherapists.

The handset, where a handset is employed for cosmetic treatments, may be in the form of a handset that includes a roller, a microdermabrasion arrangement, a serum application handset and/or a microneedle array. The liquid to be dispensed from the device of the present invention may be dispensed and applied to a patient in combination with a compressed gas, which may be an oxygen-enriched gas and it may be from the same source as the gas used to increases the dissolved oxygen in the liquid. The liquid may be applied directly as a liquid, or it may be atomised to form a mist before application to the skin. Where the handset is employed to dispense the liquid for another purpose, a simply dispensing mechanism may be adopted, for example, a dispensing gun wherein a button can be depressed to release to open an outlet, thereby allowing the dispensing of the liquid.

The device may comprise more than one means for applying the cooled, oxygenated liquid(s) to the skin of a person. This provides a more flexible treatment device, by allowing liquid(s) to be applied in different manners using a single device. If there are a plurality of containers, the respective containers may be provided with respective dispensing arrangements, or they may converge to a single dispensing handset. Thus, the containers may contain different liquids, such as a cleanser, a skin peel and a microdermabrasion liquid, each of which can, preferably, be dispensed through a respective handset or, alternatively, through a handset that is common to more than one liquid. The application of each of the liquids may be undertaken sequentially, optionally, with light therapy between the application of the liquids in the sequence in order to reduce the risk of inflammation.

The present invention extends to a method of dispensing a cooled, oxygenated liquid from a device as set out herein and, optionally, applying that dispensed liquid to a portion of a person's skin. Thus, the liquid in the container, which has an increased concentration of oxygen, can be applied topically to the skin of a person.

Thus, the method set out herein dispenses a liquid having a high level of oxygen that can then penetrate through the skin surface of a person's skin without requiring high-pressure oxygen to be applied directly to the skin, thereby improving circulation and cell regeneration within the person, which may provide an improved cosmetic treatment.

It will be appreciated that the features of the device set out in herein may be used in the dispensing of the liquid, which may include the use of a compressed gas to dispense the liquid, thereby further improving the oxygen penetration that can be achieved when using the device of the present invention.

An embodiment of the present invention will be described with reference to the following examples and figures, in which:

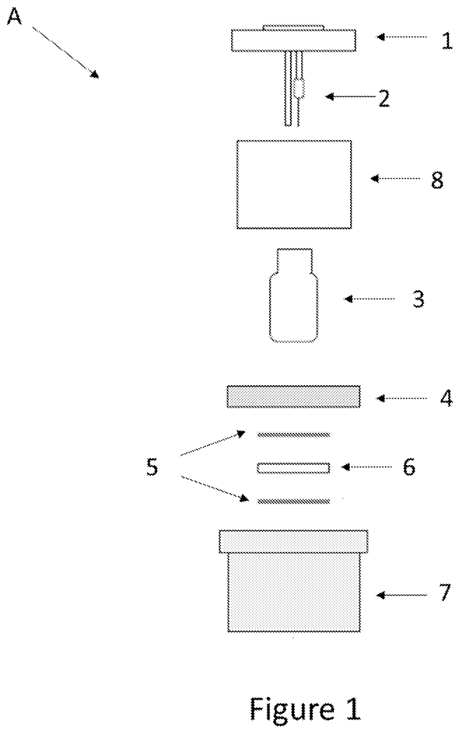

FIG. 1 illustrates an expanded view of part of a cosmetic device according to one embodiment of the present invention;

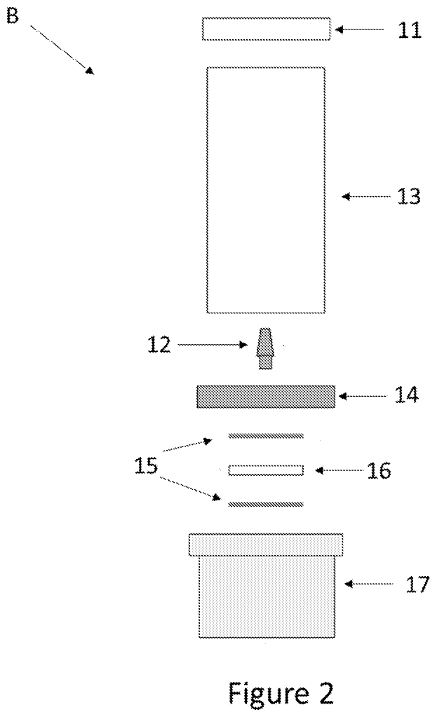

FIG. 2 illustrates an expanded view of part of a cosmetic device according to another embodiment of the present invention; and

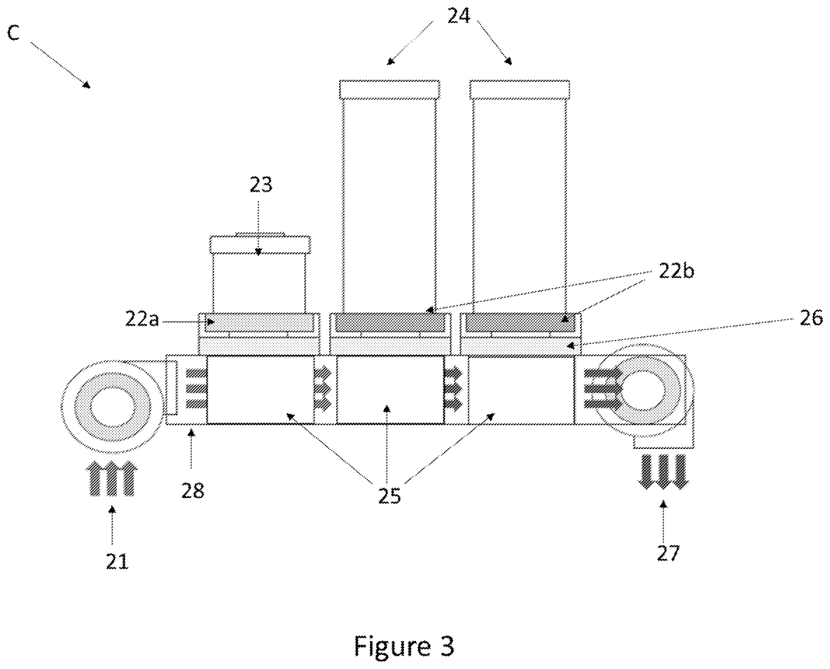

FIG. 3 illustrates a schematic diagram of another cosmetic device according to the present invention.

Referring to FIG. 1, part of a cosmetic skin treatment device A according to one embodiment of the invention is shown. The cosmetic skin treatment device includes a container 3 for holding a liquid, for example a serum or a gel, prior to dispensing from the device A, which may be in the form of application of the liquid to a person's skin. The liquid inside the container (3) is cooled by cooling apparatus and oxygenated prior to application on the user's skin. By “oxygenated”, it is meant that the concentration of dissolved oxygen within the liquid is increased.

In the exemplified embodiment, the cooling apparatus includes a water bath 8, a cooling block 4, a thermoelectric cooler module 6, two thermally conductive gap fillers 5 and a heat sink 7. The thermally conductive gap fillers 5 on either side of the cooling block 4 minimise assist with insulation and increase heat transfer from the liquid in the container 3, through the cooling apparatus to the heat sink 7.

The container 3, in which the liquid is contained, is positioned in the water bath 8 and retained in place by a container cap 1. The container 3 is screwed into the container cap by way of respective threaded sections1 and the container 3 remains suspended in the water of the water bath 8. It will be appreciated that a push-fit connection may be applied in place of the threaded connection. In this arrangement, the base of the container 3 is situated above the cooling block, being positioned around 20 mm thereabove. The liquid in the container 3 is cooled by the liquid in the water bath 8 and it is not in direct thermal contact with the cooling block 4, Thereby reducing the risk of local cool spots and producing a more even distribution of temperature therein. Although not shown, a stirring or agitating system may be employed to produce a more even distribution of temperature for the liquid within the container.

The cooling block 4 is made from a thermally conductive material, in this case aluminium to provide a good degree of heat transfer. The cooling block 4 is cooled to the extent that it freezes a portion of the water in the water bath 8 adjacent the cooling block 4, resulting in a solid ice ring up to 1 cm thick thereupon.

The cooling block 4 is in direct thermal contact with water in the water bath 8 and heat is transferred from the water bath 8, through the cooling block 4 and the thermoelectric cooling module 6 and into the heat sink 7. This heat transfer is driven by a Peltier effect in the thermoelectric cooling module 6. The heat sink 7 is attached to the thermoelectric cooler module 6 to efficiently transfer heat away from the device A. Air is blown past the heat sink 7 by a fan (not shown) in order to improve the efficiency of the heat transfer therefrom.

The liquid in the container 3 is cooled by the water bath 8 to a temperature of between 0 and 5° C., which may be between 0 and 3° C., or, preferably, between 1 and 2° C.

The container cap 1 is provided with a supply of oxygen-enriched air, which is comes from an oxygen concentrator (not shown). The oxygen concentrator concentrates oxygen from a gas supply, typically ambient air, by selectively removing nitrogen to supply an oxygen-enriched product steam. The oxygen-enriched air passes into the liquid using a gas tip 2, which is mounted in the container cap 1 and extends therefrom into the liquid within the container 3. The gas tip 2 may be any suitable gas tip, and, preferably, the gas tip is a 27-gauge stainless steel gas tip. The oxygen-enriched gas is fed to the liquid in the container 3 using the gas tip 2 to promote efficient transfer of fine bubbles of oxygen-rich gas into the liquid.

The container cap 1 additionally includes a feedline not shown for transferring the cooled, oxygenated liquid to a handset, or other dispensing outlet, via a peristaltic pump, when the device is in use.

Referring to FIG. 2, part of a cosmetic skin treatment device B according to another embodiment of the invention is shown. The cosmetic skin treatment device B includes a container 13 for holding a liquid, for example a serum or a gel, prior to application on the user's skin. The container has a cap 11, which may be a vented cap, to reduce the risk of contaminants entering the liquid and to reduce the heat transfer from the liquid therein. The liquid inside the container 13 is to be cooled by cooling apparatus and oxygenated, which is to say the concentration of dissolved oxygen therein is to be increase, prior to dispensing of the liquid from the device, which may be application on a person's skin.

In the exemplified embodiment of FIG. 2, the cooling apparatus includes a cooling block 14, a thermoelectric cooler module 16, two thermally conductive gap fillers 15, each positioned on respective sides of the cooler module 16, and a heat sink 17. The cooling block 14 is positioned to be in direct thermal contact with the liquid in the container 13 that is to be cooled. The cooling block 14 is made from a thermally conductive material, in this case stainless steel.

In this embodiment, the heat from the liquid is transferred through the cooling block 14 and the thermoelectric cooler module 16 into the heat sink 17, from which the heat is dissipated. This heat transfer is driven by a Peltier effect in the thermoelectric cooling module 16. The two thermally conductive gap fillers 15 on either side of the cooling block 14 reduce the cool loss and increase heat transfer.

The thermoelectric cooler module 16 is in direct thermal contact with the heat sink 17 to transfer heat from the cooling block 14 to the heat sink 17 to ensure the cooling block 14 remains at a low temperature to keep the liquid in the container 13 cool. Air is blown past the heat sink 17 in order to remove heat therefrom, thereby improving the efficiency of the heat transfer.

The temperature of the liquid is cooled to a temperature of between 0 and 8° C., between 2 and 7° C., or between 3 and 6° C.

The sintered gas diffuser 12 is located in the base of the container 13, at the opposite end from the cap 11, to promote an efficient transfer of fine bubbles of oxygen-enriched gas into the liquid. The sintered gas diffuser 12 covers only part of the cooling block 14 and is made of stainless steel. The oxygen-enriched gas is supplied from an oxygen concentrator (not shown).

The container 13 holds the chilled and oxygenated liquid ready for dispensing from the device, which may be by application to a patient's skin via a feedline (not shown) for transferring the cooled, oxygenated liquid to a dispensing handset, which can be via a peristaltic pump.

Referring to FIG. 3, a cosmetic skin treatment device C comprising a plurality of containers 23, 24 is shown. In this arrangement, the plurality of containers includes a first container 23 having cooling apparatus and an oxygen-enriched gas supply, similar to that shown in FIG. 1 and the further two containers 24 is each provided with a cooling apparatus and an oxygen-enriched gas supply similar to that shown in FIG. 2.

The cosmetic skin treatment device C includes an air circulation system comprising an inlet blower 21, an air duct 28 and an outlet blower 27 to help control the temperature of the liquid in the plurality of containers. The inlet blower A draws fresh ambient air into the cooling air duct 28 and passes the fresh air through the device. as shown by the direction of the arrows, to cool the heat sinks 25. The air duct 28 securely fits around the heat sinks 25 to ensure that all the cooling air passes across and through the heat sink fins, to allow efficient and effective heat dissipation.

The heat sinks 25 are attached to the thermoelectric cooler modules (not shown, but as discussed with reference to FIGS. 1 and 2) to remove heat away from the containers. The outlet blower 27 draws hot air out of the hot airway from the heat sinks 25 and out of the air duct 28 to remove any waste heat from the system, thereby helping to regulate temperature control.

In this exemplified embodiment, cooling block 22a is made from aluminium and cooling block 22b is made from stainless steel. The device further includes insulation foam 26 fitted around the cooling blocks 22a, 22b between the heat sinks 25 to insulate the cooling blocks 22a, 22b from the ambient air and to help the heatsinks 25 maintain their cooling functionality.

The cooling apparatus for the first container 23 cools a liquid to a different temperature than the cooling means of the second and third containers 24. The liquid of the first container may be cooled to a temperature of between 0 and 5° C., between 0 and 3° C., or between 1 and 2° C. The liquid of the second and/or third container may be cooled to a temperature of between 0 and 8° C., between 2 and 7° C., or between 3 and 6° C. Thus, the containers holding the liquid of a higher temperature are further along the flow of gas through the air duct 28 than the container holding the liquid of a lower temperature. This ensures efficient cooling and careful temperature regulation of the liquids.

Whilst the cooled and oxygenated liquid is intended to be dispensed directly onto a person's skin, it will be appreciated that it could be decanted into a vessel, preferably a thermally insulated vessel. In such an arrangement, it will be necessary to supply a valve on the vessel to reduce the escape of oxygen from the cooled and oxygenated liquid during the transfer. The vessel may then be used to apply the liquid to a person's skin.

The present invention has been described in relation to a cosmetic skin treatment device in the specific embodiments and in the figures, in relation to beverages and in relation to physiotherapy; however, such a device may be employed for cooling one or more containers of liquid in other fields and applications. For example, it may be advantageous to dispense liquid from the present invention in respect of scientific experiments. It will also be appreciated that the oxygen may be replaced with another gas, for example, nitrogen or carbon dioxide. This may be particularly useful in respect of the portable dispensing of carbonated drinks and/or beer

1. A liquid cooling and oxygenation device comprising:
a container for holding a liquid;
a cooling apparatus for cooling the liquid in the container to a temperature of less than 15° C.;
an arrangement to supply an oxygen-enriched gas to the cooled liquid in the container; and an outlet arrangement for dispensing the cooled, oxygenated liquid.

2. A liquid cooling and oxygenation device according to claim 1, wherein the arrangement to for supply oxygen-enriched gas comprises a gas tip and/or a gas diffuser.

3. A liquid cooling and oxygenation device according to claim 1, wherein the cooling apparatus comprises a cooling block that is in thermal contact with a water bath in which the container is positioned.

4. A liquid cooling and oxygenation device according to claim 3, wherein the cooling block is in direct thermal contact with water in the water bath.

5. A liquid cooling and oxygenation device according to claim 4, wherein the cooling block is arranged to freeze a portion of the water in the water bath adjacent to the cooling block.

6. A liquid cooling and oxygenation device according to claim 3, wherein the water bath comprises a cap on the end of the water bath opposite the cooling block and wherein the container for holding the liquid is supported by the cap.

7. A liquid cooling and oxygenation device according to claim 6, wherein the means for supplying an oxygen-enriched gas to the cooled liquid comprises a gas tip that extends into the container from the water bath.

8. A liquid cooling and oxygenation device according to claim 1, wherein the means for supplying an oxygen-enriched gas to the cooled liquid comprises a gas diffuser located within the container.

9. A liquid cooling and oxygenation device according to claim 1, wherein the cooling apparatus comprises a thermoelectric cooler module and a heat sink and wherein the heat sink is in direct thermal contact with the thermoelectric cooler module.

10. A liquid cooling and oxygenation device according to claim 9, further comprising a thermally conductive gap filler on at least one side of the thermoelectric cooler module.

11. A liquid cooling and oxygenation device according to claim 1, and further comprising a plurality of containers, each for holding a respective liquid, and each of which having cooling apparatus for cooling the liquid to a temperature of less than and each containers having a supply of oxygen-enriched gas that can be fed to the cooled liquid in each container.

12. A liquid cooling and oxygenation device according to claim 11, wherein the cooling means of a first container of the plurality of containers comprises a cooling block that is positioned to be in direct thermal contact with the container and the cooling means of the second container comprises a cooling block that is in thermal contact with a water bath in which the container is positioned.

13. A cosmetic skin treatment liquid cooling and oxygenation device according to claim 1.

14. A method of dispensing an oxygenated liquid comprising the steps of:
providing a liquid cooling and oxygenation device of claim 1;
cooling and oxygenating a liquid in the container; and
dispensing the liquid through the outlet arrangement.

15. A method according to claim 14, wherein the outlet is a handset and wherein the handset is provided with a compressed air source to dispense the liquid under pressure from the handset.
